(19) United States
(12) Patent Application Publication
(10) Pub. No.: US 2018/0161037 A1
(43) Pub. Date: Jun. 14, 2018

(54) INTERNAL ORGAN NOOSE

(71) Applicant: STEVE A FADAHUNSI, Woodford Green (GB)

(72) Inventor: STEVE A FADAHUNSI, Woodford Green (GB)

(21) Appl. No.: 15/372,870

(22) Filed: Dec. 8, 2016

(51) Int. Cl. A61B 17/12

(52) U.S. Cl. CPC A61B 17/1214; A61B 17/12099; A61B 17/12031

(57) ABSTRACT

The invention relates to a noose 1 for internal organs. Noose 1 has a tube 2 for securement to a fixed point in the body; a loop of flexible material 4 adjacent one end of the tube adapted to surround a body part adjacent one end of the tube; a slider 7 in the tube adapted to slide from a first position to a second position in the tube, said slider being of magnetic material or material attracted to a magnet; a link member 9 connecting the slider to the loop, said tube loop slider and link member being in-vivo components; and an ex-vivo controller 10 having a magnet or material attracted to a magnet adapted to cause, the magnet to slide from the first to the second position and vice versa. In use as the magnet moves from the first position to the second position so the loop is drawn into said one end of the tube so shrinking its diameter from enlarged loop to a smaller size loop thereby tightening around the part of the body it surrounds.

The current application claims a priority to the U.K. patent application serial number GB1522702.6 filed on Dec. 22, 2015.
Inventor: Steve A. Fadahunsi
Docket Number: INTERNALORGANNOOSE5768

### FIELD OF THE INVENTION

The present invention relates generally to a noose for internal organs. More specifically, firstly, a noose for the lower esophagus sphincter (LES) used to prevent acid reflux. More specifically, secondly, a noose capable of enabling an implant or a surgically operated body or organ part to be viewed ex-vivo non invasively at any time desired after the implantation surgery or after the body part or organ surgery had been done.

### BACKGROUND OF THE INVENTION

Acid reflux, also known as heartburn, is the most frequent cause of indigestion. The complication happens when the valve at the lower esophagus sphincter (LES) is abnormally relaxed and allows the stomach's acidic contents to flow back or ‘reflux’ into the gullet, causing pains and other symptoms including Barret's esophagus, esophagitis, etc. Current laparoscopic means of stopping acid reflux include Nissen Fundoplication, Enteryx (TIF) Fundoplication, Stretta, and Lynx Reflux Management System (LRMS). However, none of these procedures is free from complications. In regards to the Enteryx fundoplication, the TIF procedure, a gastrogastric wrap at the level of the gastroesophageal junction (GEJ), has been likened by critics to a slipped fundoplication, where the gastrogastric wrap is displaced to no longer support the LES. As recently as 2015, all Enteryx single pack injections were recalled because of reports that improper injection procedure can lead to serious patient injury and death. In regards to LRMS, the procedure has been linked to device erosion (device passes through esophagus wall), device failure, device migration (device does not appear to be at implant site), device removal or re-operation. In regards to Nissen Fundoplication, complications include difficulty swallowing after the operation because the stomach is wrapped too high on the esophagus or is wrapped too tightly, esophagus sliding out of wrapped position, so the LES is no longer supported (migration). Second surgeries are often inevitable, although, less successful, risky and harder to perform. In regards to Stretta, the Stretta relies on blind deployment of the radio frequency (RF) needles. The Plicator and EndoCinch require a very large overtube, which is notorious for damage to the pliable esophagus, let alone one that is inflamed or strictured.

The present invention for an internal organ noose seeks to provide an alternative to the aforementioned procedures for preventing, inter alia, acid reflux, as well as having other applications.

The present invention is sutured to a body or organ part in order to prevent sliding or dislocation of the present invention from the target location; thus to prevent the constricting filament or the constricting loop formed by the constricting filament from shifting or migrating from intended position. Further, the present invention is operable and adjustable by an ex-vivo magnet such that the present invention, even though in vivo, is able to be actuated or adjusted ex vivo when needed by the user or medical practitioner.

The present invention may also support a in vivo camera adapted to couple with an ex vivo image reader or image viewer such as an ultrasound transducer, with which it is not physically connected; whereby the in vivo camera takes in vivo images of an implant, such as the internal organ noose or a surgically operated body part, which is then read or viewed by the ex vivo image reader or image viewer. Thus, enabling an implant or a surgically operated body or organ part to be viewed ex-vivo non invasively at any time desired after the implantation surgery or after the body part or organ surgery had been done.

### BRIEF DESCRIPTION OF THE DRAWING

FIG. 1 is a top view of the present invention with the slider in the first position, wherein the constriction loop is in a diametrically expanded configuration.

FIG. 2 is a top view of the present invention with the slider in the second position, wherein the constriction loop is in a diametrically shrunken configuration.

FIG. 3 is a diagram illustrating an implementation of the present invention, wherein the present invention is positioned about the lower esophagus sphincter (LES).

### DETAIL DESCRIPTIONS OF THE INVENTION

All illustrations of the drawings are for the purpose of describing selected versions of the present invention and are not intended to limit the scope of the present invention.

The present invention is an internal organ noose. The present invention is intended to prevent, inter alia, acid reflux. For acid reflux, the present invention is situated about the patient's lower esophagus sphincter (LES) such that the present invention is able to constrict the LES. The constriction of the LES prevents contents of the stomach from entering the esophagus due to an abnormally relaxed esophageal sphincter. While the present invention is intended to be applicable about the LES of a patient for acid reflux prevention, the present invention is able to be used for applications for other organs and body or organ parts.

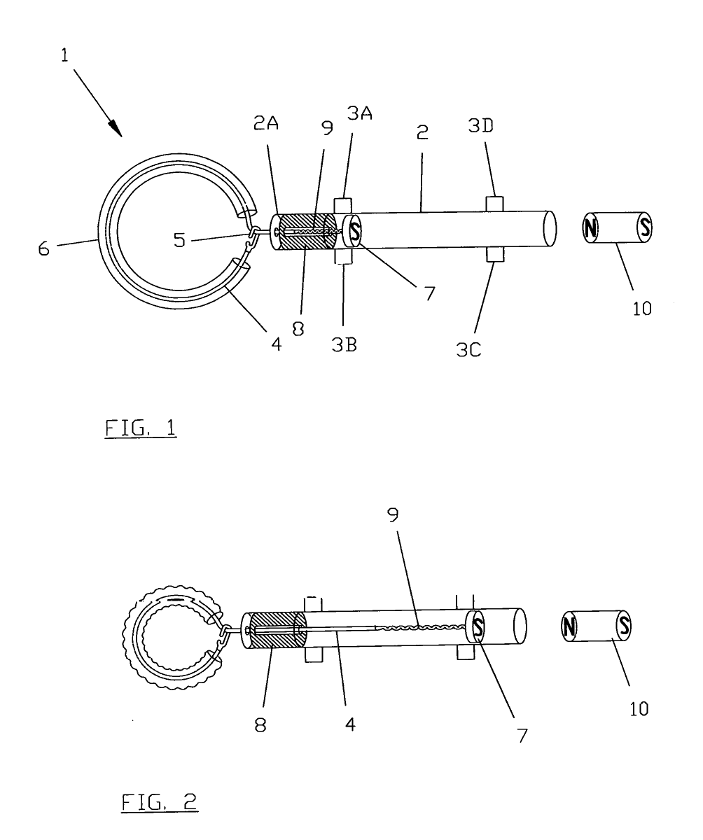

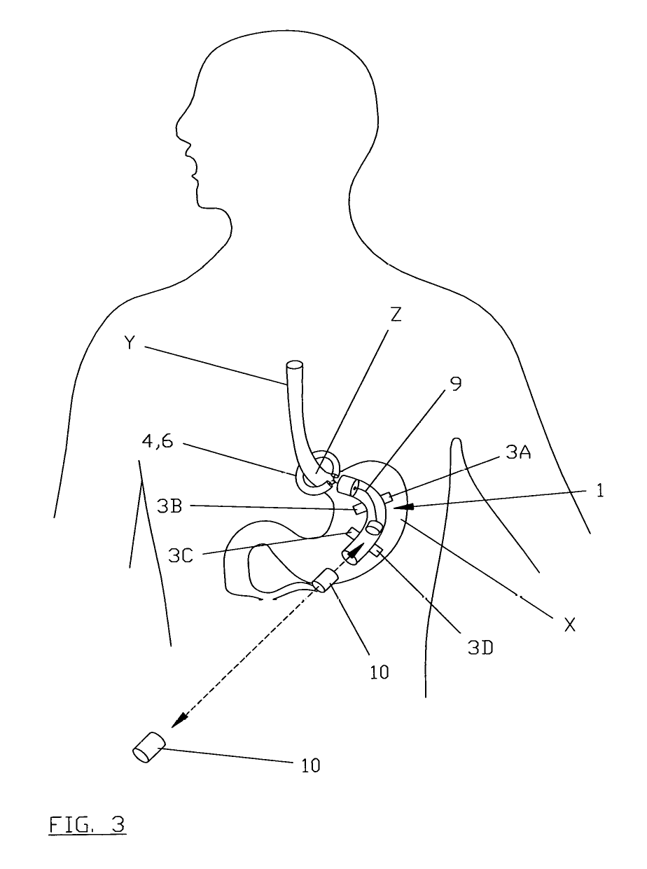

Referring to FIG. 1, FIG. 2, and FIG. 3 of a preferred embodiment; there is shown an in-vivo noose 1 for noosing internal body parts, such as the esophagus.

Noose 1 has a cylindrical mounting tube 2. Mounting tube 2 may be of flexible material and has suture mounts 3A,3B,3C,3D for securing the mounting tube to a fixed point in the body using sutures. Mounting tube 2 may be made of any suitable material usable in-vivo. Being flexible it can conform the shape of body part to which it is fixed.

A constricting filament of flexible material such as a non-elastic filament 4, with first filament end and second filament end is provided adjacent end 2A of the mounting tube and is adapted to surround and in operation thereby to constrict or noose a body part such as the esophagus adjacent the one end of the mounting tube. The constricting filament also traverses into the mounting tube with the second filament end. The first filament end of the constricting Filament 4 includes a filament attachment mechanism, such as a clip 5, which allows the constricting filament to be opened up so that it can pass around a body part and thereafter be re-connected to form a constricting loop or noose around the body part, at the said first filament end; such that the filament attachment mechanism 5 in action works much like a hangman's knot. The constricting filament of material 4 is contained in a enveloping flexible sheath of cushioning material 6 to prevent the constricting filament of material damaging a body part that it surrounds or that it is constricting.

The constricting filament 4 traverses through material 6 into mounting tube 2 while the traversing section into the mounting tube may not be enveloped in the sheath of cushioning material 6.

The second filament end of the constricting filament 4, said traversing into the mounting tube 2, comprises a slider in the form of a cylindrical magnet 7. The slider 7 is adapted to slide from a first position FIG. 1 to a second position FIG. 2 in the mounting tube 2. A slider stop 8, e.g. formed of soft tissue, prevents the magnet sliding out of mounting tube's end 2A, and serves to further anchor the constricting filament such as to prevent the constricting filament or the constricting loop formed around a body part from migrating or shifting from its position of implant.

A link member 9, FIG. 1 & FIG. 2 passes through slider stop 8 and connects the magnet 7 to the second filament end of constricting filament 4. Preferably the link member is a length of elastic material to bias the slider to the first position FIG. 1 from the second position FIG. 2 or vice versa.

The mounting tube 2 with its constricting filament 4, slider 7 and link member 9 are in-vivo components.

In accordance with the preferred embodiment, an ex-vivo controller is provided comprising, inter alia, a magnet 10. When magnet 10 is brought to a designated position on the stomach X, as in FIG. 3 close enough as in FIG. 2 to attract in vivo magnet 7 through the stomach skin, it causes the magnet to slide from the first position FIG. 1 to the second position FIG. 2. As the magnet moves from the first position FIG. 1 to the second position FIG. 2 so the elastic link member 9, at the second filament end tensions and the constricting filament is drawn into end 2A of the mounting tube, so shrinking the diameter of the constricting loop at the first filament end from enlarged FIG. 1 to a smaller size FIG. 2 thereby tightening around and constricting or noosening the part of the body it surrounds.

However, as the constricting filament's loop shrinks to a smaller size constricting loop, so also the enveloping flexible sheath of cushioning material 6 is gathered in the shrinkage thus noosening, as well as preventing any damage to the ‘LES’ at the esophagus, as depicted in FIG. 2. When magnet 10 is moved out of attraction range of magnet 7 and the magnetic attraction between the two is thus broken, then the elastic link member 9 contracts, sliding magnet 7 back further into the mounting tube 2 to the first position FIG. 1 towards the mounting tube's end 2A, and a body part squeezed by the smaller size constricting filament's loop is then unconstricted and can then expand, so expanding the diameter of the constricting loop 6 into an enlarged constricting loop again, thus to un-noose the ‘LES’ at the esophagus, FIG. 1.

As shown in FIG. 3, the noose 1 can be connected to a stomach X to tighten or loosen as required around the esophagus adjacent the lower esophageal sphincter Z. Sutures can be used to secure mounting tube to the stomach wall through suture mounts 3A,3B,3C,3D.

A wearer or medical practitioner can operate the noose, to noose and un-noose the esophagus using magnet 10 by bringing the magnet 10 close to the stomach X (ex vivo) at a designated position where it can easily attract in vivo magnet 7 from inside the mounting tube through the stomach skin, or removing magnet 10 away from attraction to magnet 7 respectively.

Alternatively, instead of removing the magnet 10, the polarity of the magnet 10 could be reversed to simply repel magnet 7. Preferably the mounting tube is positioned on the stomach in such a way that it takes full advantage of the anterior anatomical position of the stomach, such that its position is away from the rib cage cover; and at area where the stomach is best proximally exposed to the ex-vivo controller, thus to allow easy attraction and repelling between ex vivo magnet 10 and in vivo magnet 7 through the stomach skin.

A wearer can choose when to operate noose, e.g. to coincide with eating patterns.

The noose of the invention may include numerous additional features such as:

The controller may include a magnet which is an electro magnet, and also means may be provided for adjusting the strength of the electromagnet so as to adjust the size of the constricting filament's loop or constricting force imposed by the constricting filament on the constricted body part. Thus, the electromagnet allows the user, the doctor, or medical practitioner to more accurately adjust the force of constriction on the body or organ part, non invasively at any time desired after the surgery had been done.

The controller may be supported ex vivo adjacent a body part being noosed, and in a sling on the user's shoulder.

The mounting tube 2 may also be transparent so that a surgeon can easily and correctly tuck in the slider and magnet into the correct position inside mounting tube 2 during surgical implantation of the noose.

It may be possible for the slider 7 to be non-magnetic but of material attracted to magnet 10.

Alternatively slider 7 could be magnetic but the controller may be non-magnetic but of a material attracted to magnet 7.

Another embodiment of the present invention may also support a in vivo camera which coupled with a ex-vivo image reader or viewer such as a ultrasound transducer, with which it is not physically connected; can enable an implant or a surgically operated body or organ part to be read or viewed ex-vivo non invasively at any time desired after the implantation surgery or after the body part or organ surgery had been done.

This is achieved through the said supported in vivo camera being adapted to align with the lens of the ex vivo image reader or viewer, when the link member 9 is tensioned and at the FIG. 2 position; thus enabling the ex-vivo image reader or viewer's aligned lens to read or view the images captured by the in vivo camera lens with which it is aligned, through the stomach skin.

The said other embodiment comprises a camera with a lens which is preferably tubular and which preferably occupies the position in the mounting tube 2 that the soft tissue stop 8 occupies in the illustrated preferred embodiment FIG. 1 and FIG. 2.

A transparent lid made of similar material to the material of the mounting tube is provided on end 2A of the mounting tube to replace the function of the stop tissue 8 and prevent the sliding out of the slider from end 2A; as well as enable a see-through view to the filament attachment mechanism 5, the constricting loop and the esophageal wall by the said tubular camera lens, said situated at the position formerly occupied by soft tissue stop 8 in the mounting tube 2.

Thus, the said tubular camera lens preferably is also about the same size and length as the soft tissue stop 8. The tubular camera lens preferably encapsulates a tubular inner space, into which the trio of the camera's engine, a smaller tubular magnet 7, which replaces the former circular magnet 7 of the illustrated preferred embodiment, FIG. 1 & FIG. 2, as well as the elastic link member 9 all fit into and occupy. The said encapsulated tubular space inside the cameral lens thus serving as the housing for the trio to tuck away into, thus to allow a clear view of both ends of the mounting tube by the said camera lens; as well as to allow a perfect alignment to be made by the tubular camera lens with the tubular lens of the ex-vivo device when magnetically attracted to it through the stomach skin.

The tubular camera lens as well as the said trio preferably slides with, and can all-together be biased as a single unit from the first position FIG. 1 to the second position FIG. 2. In operation whereby when ex vivo magnet 10 is brought closer to the stomach X at a designated position where it can easily attract in vivo magnet 7, then the tubular camera lens and its encapsulated trio are biased from the first position FIG. 1 to the second position FIG. 2. At the second position, the tubular camera lens having encapsulated the said trio out of the way and thus able to see through to the other end of mounting tube, opposite to end 2A; is adapted to be brought into alignment with the lens of the ex vivo image reader or viewer, as the encapsulated said smaller tubular in vivo magnet 7 is attracted to ex vivo magnet 10.

Thus, allowing the in vivo tubular camera lens to be aligned as adapted with an ex-vivo apparatus's lens, also preferably a tubular lens, that can read or view through the stomach skin, any images captured by the aligned in vivo tubular camera lens.

The said trio encapsulated by the camera lens having tucked away into their said encapsulated housing allow alignment of the in vivo tubular camera lens with the ex vivo tubular lens of the image reader or image viewer through the stomach skin. Preferably another transparent lid, similar to that provided on end 2A is also provided on the opposing end of the mounting tube such that the slider may be biased right through to the tip of the said opposing end of the mounting tube 2 without the possibility of ejecting; thus to allow a perfect alignment of the in vivo and ex vivo lenses through the stomach skin.

Thereby allowing images to be read or viewed ex vivo when the slider is in the second position FIG. 2. Thus allowing vital information of implanted device, noose 1, and its affects and effects on the noosed part of the body, the esophagus, to be obtained un-invasively ex vivo at any time desired after the implantation had been done.

It may be that the noose of the invention can be used to noose body parts other than the esophagus.

The invention may take other forms different to the specifically described above.

Further modifications will be apparent to those skilled in the art without departing from the scope of the present invention.

What is claimed:

1. A internal organ noose comprising:
a mounting tube;
a constricting filament;
a filament attachment mechanism;
a slider;
a link member;
the mounting tube being adapted for securement to a fixed point in the body;
the constricting filament, comprising a first filament end and a second filament end, adjacent one end of the mounting tube, adapted to form a constricting loop to surround a body part adjacent the one end of the tube;
the filament attachment mechanism adapted to allow the constricting filament to be passed around a body part and thus be positioned to form the constricting loop round the body part;
the slider adapted to slide from a first position to a second position;
the link member adapted to connect the slider to the constricting filament, said mounting tube, constricting filament, filament attachment mechanism, slider and link member being in vivo components;
a ex-vivo controller having a magnet or material attracted to a magnet adapted to cause slider to slide from the first position to the second position thus to cause a constricted loop or a noose to be formed with the constricting filament around a body part surrounded by the constricting filament;
a flexible cushioning sheath adapted to envelope the constricting filament to prevent the constricting filament from damaging a body part it surrounds;
at least one stop on the mounting tube adapted to prevent the sliding out of the slider from the mounting tube.

2. A internal organ noose of claim 1 wherein the mounting tube is of a flexible material.

3. A internal organ noose of claim 1, whereby the constricting filament is of a flexible material and comprises a first filament end and a second filament end; whereby a filament attachment mechanism and a link member connecting the slider to the constricting filament are at the said first filament end and the second filament end respectively.

4. A internal organ noose according to claim 1, wherein the constricting filament's loop is enveloped in a sheath of cushioning material to prevent the loop from damaging a body part that it surrounds.

5. A internal organ noose of claim 1 wherein the constricting filament traverses through the enveloping flexible sheath adjacent the constricting loop and into the mounting tube.

6. A internal organ noose of claim 1, wherein the filament attachment mechanising is a filament clip.

7. A internal organ noose according to claim 1 wherein the link member includes a length of elastic material to bias the slider to the first position from the second position or vice versa and wherein the slider is a magnet or material attracted to a magnet.

8. A internal organ noose according to claim 1, wherein the said at least one stop is at one end of mounting tube and is formed of soft tissue material.

9. A internal organ noose according to claim 1, wherein the said at least one stop is at one end of the mounting tube and is formed of flexible transparent material.

10. A internal organ noose according to claim 1, wherein there are stops at both ends of the mounting tube, both of which are formed of transparent material.

11. A internal organ noose according to claim 1 wherein the at least one stop prevents the sliding out of content of the mounting tube.

12. A internal organ noose, according to claim 1 wherein the filament attachment mechanism is a adapted knot.

13. A internal organ noose as claimed in claim 1, comprising of:
at least one suture mount; the at least one suture mount being connected to a body part to secure and stabilise the mounting tube to and on the body part, thus to prevent migration or shifting of constricting filament and thus the constricting filament's loop from its intended position on the body part being constricted or noosed.

14. A internal organ noose according to claim 1, wherein sutures are adapted as its securement to another part of the body.

15. A internal organ noose according to claim 1, wherein connection points are adapted to secure the mounting tube to the stomach.

16. A internal organ noose according to claim 1, wherein a constricting filament is adapted to engage the Lower Esophagus Sphincter and to stop acid reflux.

17. A internal organ noose, as claimed in claim 1, wherein the controller includes a magnet which is an electromagnet, and means may be provided for adjusting the strength of the electromagnet so as to adjust the size of the constriction loop thus the constricting force imposed by the constricting loop on a body part.

18. A internal organ noose according to claim 1, wherein the controller may be supported ex vivo adjacent or near a body part being noosed, or in a sling on the user's shoulder or adapted to a user's need.

19. A internal organ noose according to claim 1, wherein the noose may also support a camera and ultrasound transducer to take images of the body part being noosed.

20. A internal organ noose according to claim 1 wherein the noose may support a in vivo camera coupled with an ex vivo image reading device or image viewing device, with which it is not physically connected to enable an implant or a surgically operated body or organ part to be viewed ex-vivo non invasively at any time desired after the implantation or after the body part or organ surgery had been done.
